(19) United States
(12) Patent Application Publication
(10) Pub. No.: US 2021/0008202 A1
(43) Pub. Date: Jan. 14, 2021

(54) Using Paper with Heightened KELEA to Enhance the Alternative Cellular Energy (ACE) Pathway

(71) Applicant: William John Martin, South Pasadena, CA (US)

(72) Inventor: William John Martin, South Pasadena, CA (US)

(21) Appl. No.: 16/508,255

(22) Filed: Jul. 10, 2019

(51) Int. Cl. A61K 41/00; A61K 9/00

(52) U.S. Cl. CPC A61K 41/0004; A61K 9/0014

(57) ABSTRACT

Placing paper onto the skin of an individual is used to support the alternative cellular energy (ACE) pathway in a human or animal subject. The benefits can be increased if the paper is first provided with a heightened level of KELEA by being stored for several hours in the vicinity of containers of KELEA activated water. KELEA is an abbreviation of “kinetic energy limiting electrostatic attraction.” The ACE pathway is a form of cellular energy, which is different from that provided by the calories in food or, in the case of plants and certain bacteria, by photosynthesis. In relative terms, the direct contact of the body with paper that has a heightened level of KELEA achieves better transfer of KELEA to living life forms than does the indirect exposure of the body to KELEA activated water contained in wearable waterceutical pouches. The direction of transfer of KELEA between the skin of an individual and paper can also be used to monitor the activity of the ACE pathway in the individual. Paper with heightened KELEA has numerous therapeutic and increased wellbeing applications in humans, animals, and plants.

### RELATED PUBLICATION BY THE APPLICANT

1. Martin, W. J. (2003) Stealth Virus Culture Pigments, A Potential Source of Cellular Energy. Experimental Molecular Pathology, 74, 210-223.
2. Martin, W. J. (2005) Alternative Cellular Energy Pigments from Bacteria of Stealth Virus Infected Individuals. Experimental Molecular Pathology, 78, 217-217.
3. Martin W J (2014). Stealth Adapted Viruses; Alternative Cellular Energy (ACE) & KELEA Activated Water. Author House, IN. pp 321. ISBN 978-1-4969-0496-6.
4. Martin, W. J. (2014) KELEA Activated Water—Enhancing the Alternative Cellular Energy (ACE) Pathway. In Stealth Adapted Viruses; Alternative Cellular Energy (ACE) & KELEA Activated Water. Author House, Bloomington, Ind., USA, pp. 115-144.
5. Martin, W. J. (2014) KELEA Activated Water Leading to Improved Quantity & Quality of Agricultural Crops. Advances in Plants & Agriculture Research, 2, 00033.
6. Martin, W. J. (2015) Therapeutic Potential of KELEA Activated Water. International J of Complementary & Alternative Medicine, 1(1), 00001.
7. Martin, W. J. (2015) Alternative Cellular Energy Pathway Therapy Using KELEA Activated Water. International J Complementary & Alternative Medicine, 2(2), 00051.
8. Martin, W. J. (2015) KELEA, A Natural Energy That Seemingly Reduces Intermolecular Hydrogen Bonding in Water and Other liquids. Open Journal of Biophysics, 5, 69-79.
9. Martin, W. J. and Laurent, D. (2015) Homeopathy as A Misnomer for Activation of the Alternative Cellular Energy Pathway, Evidence for the Therapeutic Benefits of Enercel in a Diverse Range of Clinical Illnesses. International J Complementary & Alternative Medicine, 2(1), 00045
10. Martin, W. J. (2015) Interacting Light Paths Attract KELEA (Kinetic Energy Limiting Electrostatic Attraction) and Can Lead to the Activation of Water. Open Journal of Biophysics, 5, 115-121.
11. Martin, W. J. (2015) Interactive Electric Fields Can Attract KELEA (Kinetic Energy Limiting Electrostatic Attraction) and Can Lead to the Activation of Water. International J Complementary & Alternative Medicine, 1(6), 00034.
12. Martin, W. J. (2016) KELEA (Kinetic Energy Limiting Electrostatic Attraction) May Add to the Measured Weight of an Object. J Modern Physics, 7(6), 461-472.
13. Martin, W. J. (2016) KELEA (Kinetic Energy Limiting Electrostatic Attraction) Offers an Alternative Explanation to Existing Concepts Regarding Wave-Particle Duality, Cold Fusion and Superconductivity. J Modern Physics, 7(15), 1995-2007
14. Martin, W. J. (2016) Preparing and Using KELEA Activated Water to Enhance the Alternative Cellular Energy (ACE) Pathway in the Therapy of Multiple Illnesses. International J Complementary & Alternative Medicine, 3(1), 00059.
15. Martin, W. J. (2015) KELEA Activation of Water and Other Fluids for Health, Agriculture and Industry. Journal of Water Resource and Protection, 7, 1331-1344.
16. Martin, W. J. (2016) KELEA, Cosmic Rays, Cloud Formation and Electromagnetic Radiation, Electropollution as a Possible Explanation for Climate Change. Atmospheric and Climate Sciences, 6(2), 174-179.
17. Martin, W. J. (2016) Cancer as an Insufficiency of Cellular Energy (ICE), Therapeutic Approaches Based on Enhancing the Alternative Cellular Energy (ACE) Pathway. International J Complementary & Alternative Medicine, 3(3), 00074.
18. Martin, W. J. (2016) Insufficiency of Cellular Energy (ICE) in Neurons, From Electrical Hyperactivity to Quiescence. International Journal Complementary Alternative Medicine, 4, 00118.
19. Martin, W. J. (2016) Insufficiency of Cellular Energy (ICE) May Precede Neurodegeneration in Alzheimer's Disease and Be Treatable via the Alternative Cellular Energy (ACE) Pathway. Advances in Alzheimer's Disease, 6(1), 1-12.
20. Martin, W. J. (2016) Insufficiency of Cellular Energy (ICE), The Basis for Many Illnesses Potentially Correctable Using KELEA Activated Water. International J Complementary & Alternative Medicine, 4(1), 00106.
21. Martin, W. J. (2017) Cancer Is Treatable via the Alternative Cellular Energy (ACE) Pathway, Journal of Cancer Therapy, 8, 1279-1290
22. Martin, W. J. (2017) Using KELEA (Kinetic Energy Limiting Electrostatic Attraction) to Improve the Efficiency of Fuel Combustion. Open Journal Air Pollution, 6(3), 103-116.
23. Martin, W. J. (2017) Is KELEA (Kinetic Energy Limiting Electrostatic Attraction) a Source of Chemical Energy? MOJ Biorg Org Chem, 1(2), 54-58.
24. Martin, W. J. (2017) Insufficiency of Cellular Energy (ICE) from the Alternative Cellular Energy (ACE) Pathway Limiting the Specialized Functions of Neuronal Cells. International J Complementary & Alternative Medicine, 4(2), 00112.
25. Martin, W. J. (2017) Hyper-Excitability Followed by Functional Quiescence in Neuronal Cells Caused by Insufficient Cellular Energy (ICE), Treatable by Enhancing the Alternative Cellular Energy (ACE) Pathway. World Journal of Neuroscience, 7(3), 257-266.
26. Martin, W. J. (2017) Tissue Regeneration without Scarring Achieved by Enhancing the Alternative Cellular Energy (ACE) Pathway. Journal of Cosmetics, Dermatological Sciences and Applications, 7(1), 82-98.
27. Martin W J (2018) Is the placebo effect mediated by the Alternative Cellular Energy (ACE) pathway? International J Complementary & Alternative Medicine, 11(4): 231-233.
28. Martin W J (2018) KELEA Activated Water as an Alternative to Stem Cell Injections in Regenerative Medicine. International J Complementary & Alternative Medicine, 11(5), 251-258.
29. Martin W J (2019) Electromagnetic Radiation Causes Weight Loss and Weight Destabilization of Objects with Presumed Elevated Levels of KELEA (Kinetic Energy Limiting Electrostatic Attraction), Relevance to Human Health and to Global Warming. Modern J Physics 10(3), 195-213.

### RELATED PATENT APPLICATIONS BY THE APPLICANT

ACE-pigments and Humic acid as energy sources. Application Ser. No. 10/192,936 Method of assessing and of activating the alternative cellular energy (ACE) pathway in the therapy of diseases. William John Martin Submitted Jan. 16, 2008. Application Ser. No. 12/009,195

Enerceutical mediated activation of the alternative cellular energy (ACE) pathway in the therapy of diseases. Application Ser. No. 12/151,779

Regenerative wound healing using copper-silver citrate composition. Application Ser. No. 12/288,749

Enerceutical activation of the alternative cellular energy (ACE) pathway in therapy of diseases. Application Ser. No. 12/069,597

Method of using the body's alternative cellular energy pigments (ACE-pigments) in the therapy of diseases application Ser. No. 12/378,934

Urine as a source of alternative cellular energy pigments (ACE-pigments) in the assessment and therapy of diseases. Application Ser. No. 12/381,003

Activation of the alternative cellular energy (ACE) pathway in the therapy of diseases. Application Ser. No. 12/802,605

Methods for the detection of alternative cellular energy (ACE) pigments and for monitoring of the ACE pathway in the diagnosis and therapy of diseases. Application Ser. No. 12/802,763

Diagnostic value of systemic ACE pathway activation in the detection by fluorescence of localized pathological lesions. Application Ser. No. 12/804,619

Enerceutical mediated activation of the alternative cellular energy (ACE) pathway in the therapy of diseases. Application Ser. No. 12/151,779

Energy Charged Liquids to Enhance Enerceutical Activation of the Alternative Cellular Energy (ACE) Pathway in the Therapy of Diseases. Application Ser. No. 12/972,344

Energy Charged Alcoholic Beverages for Enhancing the Alternative Cellular Energy Pathway in the Prevention and Therapy of Diseases. Application Ser. No. 12/984,582

Energy Charged Liquids to Enhance Enerceutical Activation of the Alternative Cellular Energy (ACE) Pathway in the Therapy of Diseases application Ser. No. 12/972,344

Methods for Detecting and Monitoring the Activity of Energized Water and Other Liquids Useful for Enhancing the Alternative Cellular Energy Pathway in the Prevention and Therapy of Diseases. Application Ser. No. 13/016,948

Methods for Increasing the Kinetic Activity of Alcohol, Water and Other Liquids, so as to Render the Liquids More Useful in Enhancing the Alternative Cellular Energy Pathway in the Prevention and Therapy of Diseases. Application Ser. No. 13/029,116

Methods for Increasing the Kinetic Activity of Alcohol, Water and Other Liquids, so as to Render the Liquids More Capable of Enhancing the Alternative Cellular Energy Pathway in the Prevention and Therapy of Diseases application Ser. No. 13/040,262

Methods for Increasing the Kinetic Activity of Water and Other Liquids, so as to Render the Liquids More Useful in Enhancing the Alternative Cellular Energy Pathway and in Various Other Agricultural and Industrial Applications. Application Ser. No. 13/166,800

Use of Plants Extracts to Activate Water, Alcohol and Other Liquids. Submitted Oct. 27, 2011. Application Ser. No. 13/272,215.

Methods of Transferring Energies to Water, Alcohols and Minerals. Submitted Nov. 25, 2011. Application Ser. No. 13/304,558.

Use of Certain Foods and Dietary Supplements as Water and Beverage Activating Enerceuticals. Application Ser. No. 14/507,822

Heat as a Method to Enhance the Fluid Activating Ability of Humic Acids, Zeolites and related Enerceuticals. Application Ser. No. 14/294,076

Method of Enhancing the Alternative Cellular Energy Pathway in Humans and Animals Using Wearable Items That Contain KELEA Activated Water. Submitted Feb. 18, 2019. Application Ser. No. 16/278,712

Method of Detection and Measurement of a Life Force Energy, Also Known as KELEA in Liquids & Other Materials. Submitted May 23, 2019. Application Ser. No. 16/421,344

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not Applicable

### FIELD OF THE INVENTION

The invention is within the field of energy-based medicine. It relates to methods for providing humans and animals with a type of cellular energy, which is different from the energy obtained from the metabolism of the calories in food. The Applicant has described the non-food-derived cellular energy as the alternative cellular energy or the ACE pathway. The primary source of energy for the ACE pathway is from the environment. This energy is designated by the Applicant as KELEA, being an abbreviation for kinetic energy limiting electrostatic attraction. KELEA is an extension of the somewhat vague concepts alluded to by many ancient societies with terms such as chi, prana, universal life force, spirit, etc. Apart from sun-driven photosynthesis, life force energies beyond food metabolism are not currently acknowledged by mainstream biologists. This is in spite of the growing acceptance that certain types of “activated” water may intrinsically provide useful biological benefits when they are consumed by humans and/or when used in agriculture. The Applicant has plausibly integrated the concepts of KELEA, ACE pathway, and activated water. Specifically, he has proposed that humans and animals attract KELEA into the body as a means of adding an additional dynamic or kinetic quality to the body's fluids. This added energy can be converted into chemical energy and used in other ways, which collectively comprise the body's ACE pathway. Based on this formulation, method for delivering additional KELEA to life forms can be beneficial, especially for life forms with impaired cellular functions due to an insufficiency of cellular energy.

### BRIEF SUMMARY OF THE INVENTION

Earlier discoveries by the Applicant have supported the therapeutic uses of activated water as a means of enhancing the ACE pathway in humans, animals, and plants. These discoveries are partially detailed in the listed earlier patent applications and largely reported in the above cited published references. These cited earlier patent applications and publications are incorporated herein into this application. As disclosed in patent application Ser. No. 16/278,712, the activated water can be conveniently provided and used in the form of “wearable waterceutical pouches” applied to the surface of the body.

KELEA activated water containing pouches are currently being prepared using polyvinyl chloride (PVC) mini hot water bags of 100 ml capacity and measuring 12×11 centimeters. The bags have a screw top. Such bags are produced in Hong Kong by a company called Dragon Sonics. Similar bags are also available from several Chinese manufactures and distributors. The bags are available from Amazon and at lower prices from other suppliers. From 50-60 ml of activated water are added to each bag. Initially, the screw top was reapplied to the bag and sealed with the addition of a PVC pipe cement, such as Christy's Red Hot Blue, T plus 2 Sealant, or Rectorseal epoxy putty. Pipe thread sealant tape has been similarly used. The sealing was occasionally incomplete. Moreover, the screw top area of the bag was somewhat uncomfortable when the bag replaced by using a “seal-a-meal” device to heat seal and separate the lower water containing section of the bag, from the upper area of the bag with the screw top. The heat sealing and detachment of the top and bottom portions of the bag occurs within 60 second of pressing down onto the water containing bag using the seal-a-meal device. The KELEA activated water containing bags are now far more convenient to use as a “wearable waterceutical pouchs” and for other purposes.

An important quality assurance development in the use of these pouches has been the discovery that KELEA activated water in the pouches can increase the measured weight of cellulose containing materials, including wood, cotton fabrics and paper, which are placed close to the pouches. The testing provided evidence that the water in the pouches is retaining its activity, even after several months of use and/or storage. The increase in weight of the cellulose containing materials is a reliable indication that if water is sufficiently activated, it can continually attract and release measurable KELEA, possibly associated with oscillations of the water molecules. Some of the released KELEA from the activated water is able to attach to the separated electrical charges on cellulose molecules, especially in the shorter, branched, interlocking form of cellulose that occurs in paper. As recently published, KELEA added to the cellulose materials can be subsequently stripped by electromagnetic radiation generated by the electronic balance, or by other devices. As explained above, the levels of the reversible weight increase by a single rolled sheet of regular typing paper placed onto or near to a KELEA activated water bag is being used to confirm the retention of KELEA radiating activity of the KELEA activated water containing bags over several months of use and/or storage.

The change in weight of a rolled single sheet of paper has also been used to assess the KELEA levels in individuals. An 8.5″ high rolled sheet of regular 8.5″×11″ writing paper can be held in this shape using a small strip of adherent tape, rubber band, or metal clip. A single sheet of writing paper will typically weigh about 4 and a half grams. The weight of the same sheet of rolled paper can, however, change within a range of 50 mg (˜1%), depending upon its surroundings. Consistently, the weight will increase by placing the paper nearby to KELEA activated water and will decrease by subsequently exposing the rolled sheet of paper to a source of electromagnetic radiation. Holding a rolled sheet of paper in the hands or pacing the rolled sheet of paper onto the skin of the body can also directly affect its weight. An increase in weight is interpreted as the transfer of KELEA from the body to the paper. A decrease in weight is interpreted as the transfer of KELEA from the paper to the person holding the paper. The test can be better quantified by using both paper with a heightened level of KELEA from having been stored near activated water and paper that is deficient in KELEA from having been placed for several hours in an area of high electromagnetic radiation. It is easier to show weight reductions with the former and to show weight increases with the KELEA heightened and KELEA deficient paper, respectively.

These observations led to the possibility that KELEA enhanced paper could be substituted for KELEA activated water as a means of delivering KELEA to a person, and by inference to animals and plants. This has turned out to be clearly so with the conclusion that paper with an enhanced level of KELEA is more effective as a KELEA delivering mechanism than the direct use of KELEA activated waterceutical pouches. This discovery will greatly facilitate the widespread therapeutic uses of KELEA in humans, animals, and plants.

### BRIEF DESCRIPTION OF THE DRAWINGS

Not Applicable and none included

### DETAILED DESCRIPTION OF THE INVENTION

Approximately two gallons of KELEA activated water in numerous 2 oz bottles and in several larger containers have been stored in the same room as an electronic balance. The water has been variously activated using magnesium oxide pellets (prills), humic acid, zeolites, Brown's gas, and miscellaneous other methods. In separate studies KELEA activated ethanol in the form of EverClear (75% ethanol) and Stroh's rum (80% ethanol) with added humic acid (0.1%) have been used to add KELEA to nearby paper. So too can various terpene containing solutions, such as bottles of the d-limonene household cleaning product called Orange-TKO, (Orange-TKO Industries, Calgary, Alberta, Canada.

To conduct the experiments described in this application, a case of 24-bottles of a commercially available supply of activated water was obtained from Starchamber Products, Inc. CA. It placed into a different room than the above-mentioned solutions. Several cases of Heal+H₂O brand of drinking water from Ecuador were also obtained from Springfield Farms, N.C. and placed into another room. These cases of water were additionally exposed for 24 hours to an energy enhancement system, comprising sets of inwardly facing computers screens displaying scrolling lines of symbols. The ever-changing images from the computer screens converge to a central area in the room. The convergence of photon evoked positive and negative charges is presumably acting in a manner to attract additional KELEA, such that the overall level of KELEA is increased, with some of the KELEA moving into the drinking water. A third tested water comprised twenty 50 ml bottles of a supposedly homeopathic product called Enercel that is available from World Health Advanced Technologies, Sarasota, Fla. In essence, these three products are all good examples of KELEA activated water. These sources are not unique, however, since some store purchased water can be significantly activated. The tests for activation include weight loss due to enhanced vaporization in closed but not completely sealed containers; the linear dissolving of sprinkled neutral red dye, including the penetration of the surface due to lowered surface tension; and the vibration and attraction and repulsion of small particles of Lidocaine sprinkled onto the surface of the water. The most relevant testing, however, has been showing an increase in the weight of single or multiple sheets of paper that are placed close to the unopened containers of the water. Each grouping of products was able to significantly increase the measured weight of regular paper placed in close proximity to the products.

It should also be noted that the ability to of containers of KELEA activated water to transfer KELEA to nearby paper is increased by a series of jolts to the containers. This is equivalent to the process of succession of homeopathic solutions. It can be applied to multiple bottles of unopened water in a carrying case. A suitable approach is to lift the case to about a foot and then to drop the case so that the bottles are jolted when hitting the ground. This can be done several times before pacing the paper to be energized into the case with the KELEA activated water. Jolting is a not a requirement for KELEA transfer to the paper, but it increases the efficiency of the process.

The paper that has been KELEA enhanced using this approach has included single and multiple sheets of 8.5″×11″ writing paper. One source for the writing paper has been Hammermill Copy plus, 20 lb., 92 brightness. One, 6, 11, and 16 sheets of this paper were rolled into 8.5″ high columns and held in place using either tape or a rubber band. Other sources of paper were rolled magazines and a rolled copy of the Los Angeles Times newspaper. The different examples of the paper were either tested individually or in groups to confirm the following: That the weigh would vary depending on whether the item was positioned nearby to any of the above sources of KELEA activated water or placed onto the surface of the body. The ranges of weight measurements variability of the different items, which were dependent upon the location of the paper, were approximately 1% of the lowest weight of the particular item. For examples over a series of over 10 measurements performed over several days, the ranges of the weights obtained with the rolled 1, 6, 11, and 16 sheets of writing paper were 4.7336 grams to 4.6833 grams; 28.2636 grams to 27.9168 grams; 49.1860 grams to 48.7744 grams; and 93.9115 grams to 92.8825 grams, respectively. The percentage increases in weight compared to the lowest recorded levels for each item were 1.1%, 1.2%, 0.8%, and 1.1% respectively. The weight of the rolled Los Angeles Times newspaper in 15 measurements over a week ranged from 163.6569 grams to 165.2126 grams, a difference of 0.95%. The highest weight for each item of paper was achieved by storing the item in the case with jolted bottles of KELEA activated water. The lowest weights were achieved by placing the item next to the skin of an individual throughout the night. The increases in weight of the paper in the vicinity of activated water were near maximum within approximately 2 hours of exposure. The decreases in weight in the paper that was placed onto the skin were also initially rapid but were also sustained at slower rates for prolonged periods. One experiment was continued for greater than 12 hours giving the impression of some ongoing external absorption of KELEA was offsetting the transfer of KELEA to the body. In several repeat experiments, the direct contact of the paper with the skin was more effective in achieving the transfer of KELEA than when using intervening materials, such as aluminum foil. The weight-losing effect could still be demonstrated, however, especially if the intervening material was thin Saran™ wrap or a Ziploc™ bag. Still, the preference is for direct skin contact with the paper.

Removing the paper with heightened KELEA from the vicinity of the activated water to a third or different location did not result in a meaningful reduction in its weight over several hours. In contrast, placing a paper item with reduced weight from being close to an individual, into the general environment lead to a steady increase in its weight, but not to the level achieved when the item is placed in close proximity to the activated water. Continuing to weigh the rolled sheets of paper or the newspaper with increased weight in the electronic balance led to the gradual decline in the weight of the item, which is attributed to the electromagnetic radiation emitted by the electronic balance. More interesting, continuing to weigh the rolled sheets of paper or the newspaper after their weight had been dramatically reduced by contact with the body led to a steady increase in the item's weight. This observation is consistent with another finding that the reduction in weight of an item caused by skin contact is significantly greater than that achievable by exposing the item to electromagnetic radiation either emitted by the electronic balance and/or by the speakers of a turned-on radio. Electromagnetic radiation presumably carries a certain amount of KELEA. The amount of KELEA carried by the electromagnetic radiation can seemingly be increased by contact with paper with a heightened level of KELEA or can be diminished by contact with paper that is deficient in KELEA. Similar to heat transfer, the flow of KELEA between items may be determined by the existing levels coupled by the relative attraction of KELEA. It would seem that KELEA deficient paper can gain some KELEA from the KELEA associated with electromagnetic radiation.

The same bilateral transfer of KELEA can apparently apply to individuals. Thus, paper that is deficient in KELEA can be a sensitive measure to detect even a relatively low level of KELEA in an individual, as evidenced by a slight increase in weight of the paper. If there is no increase in weight, the person can be assessed as having an even lower level of KELEA. Conversely, if a person can further increase the weight of paper with heightened KELEA, or even if the weight remains the same, it can be concluded that he or she has an abundance of KELEA. A reduction in weight of paper with a heightened level of KELEA is an indication of potential added benefit from longer term contact with paper with a heightened level of KELEA. For this purpose, multiple sheets of paper, newspapers, and other large paper items with heightened KELEA are potentially able to transfer more KELEA than smaller quantities of paper. These need for larger quantities of paper with heightened KELEA may be offset, however, if the paper placed close to the body can continue to receive additional KELEA from the environment. This can be more efficiently accomplished by having nearby KELEA activated water. The benefit of these approaches to particular individuals can be monitored using the above mentioned KELEA assessment assay using the changes in weight of single sheets of paper that have either deficient or heightened levels of KELEA.

A personal comparison has been made over the last several months of the relative benefits of using KELEA activated water as a wearable waterceutical pouch or applying KELEA sheets of paper with heightened levels of KELEA to the skin. The former had generally consisted of wearing two pouches of 50 ml Enercel solution within the socks. Various different pouches would be worn during the day and also at night. This approach was contrasted with the benefits of simply placing one or more rolls of multiple sheets of paper with heightened KELEA within the pajamas while overnight sleeping. The level of KELEA transfer was monitored by weighing the paper items before going to bed and upon waking. The paper items were recharged during the day by being placed in close proximity to one or other of the groupings of unopened bottles of KELEA activated water. By various criteria, including a sense of wellbeing, awareness, work output, quality of sleep and blood pressure stability, the use of the rolled paper has been more effective than the use of the water patches. What is most impressive is the far greater extent that the body can reduce the relative weight of paper with heightened KELEA (approximately 1%) than the minor observed reduction in the weight of the used sealed bag containing the KELEA activated water. Paper with heightened KELEA has now also be used as a wearable device in the form of variously sized pads. Individual or multiple sheets of paper can be easily strapped to different parts of the body or folded into a bandage type arrangement to be applied to painful areas or areas of local pathology. The prospect of locally treating a herpes outbreak using paper with heightened KELEA is awaiting the identification of a suitable volunteer and Institutional Review Board (IRB) approval.

Paper sheets with heightened KELEA have also been attached to the collars of a high energy Vizsla dog and a more sedentary Pit bull dog. Somewhat surprisingly, both dogs reduced the measured weights of the sheets of paper. One of the dogs had been drinking KELEA activated water. A newspaper with heightened KELEA has now been added to the kennel of this dog.

Other studies are underway, including survival studies on single cell organisms recovered from a vase of wilting flowers. The consistent indications are that the paper with heightened levels of KELEA can provide substantial biological benefits to life forms. Moreover, as an aside, the transfer of KELEA from paper, including newspapers, to plants probably occurs in the use of paper as mulch in farming. By using paper-derived mulch with KELEA activated water is likely to be more useful than using either approach alone in farming operations. Plans to undertake such studies are underway.

Overall, the data are consistent with a hierarchical preferred direction of transfer of KELEA. Specifically, humans, dogs and presumably all other living creatures are more receptive to KELEA from cellulose containing materials, and in particular, from paper with a heightened level of KELEA, than they are to KELEA coming indirectly from KELEA activated water. Yet KELEA activated water is apparently more effective in transferring KELEA to paper than the transfer of KELEA from paper to regular water. That is, although it does occur, paper with heightened KELEA is a relatively inefficient way of transferring KELEA to other fluids outside of the body, when compared to the use of various water activating compounds, or activated water by itself. For this reason, the pouches of KELEA activated water are still finding good use in the transfer of KELEA to other fluids, including water and both gasoline and diesel fuels.

Personal experiences using the above procedures are also in agreement with a fundamental benefit of working towards increasing the levels of KELEA as a means of improving higher level brain functions. This improvement may in turn be increasing the KELEA receiver function of the brain. As discussed in many of the cited publications, increased KELEA can also help compensate for numerous medical conditions attributed to an insufficiency of cellular energy. The approach described in this Application can also be scaled up to include the use of paper gowns, paper bed sheets, paper mattress covers, etc., with heightened levels of KELEA Tightly rolled sheets of paper are convenient in the sense that they can be placed vertically upon the weighing pan of electronic balances with a sliding glass top cover. The range of approximately 1% in weigh change is also a rather consistent measure of difference of paper with depleted and with heightened KELEA.

As an example of a specific comparison between KELEA activated water and paper with heightened KELEA, an individual with hearing difficulties has been instilling Enercel mist spray into his ear canals. He has since replaced this approach by using a roll of paper pad with heightened KELEA over the ear with seemingly greater improvement in hearing than when using Enercel. Such studies are awaiting Institutional Review Board (IRB) approval.

The findings uncovered by this research are interesting in the context that cotton clothing. Cotton fabrics are primarily composed of cellulose and are widely used in clothing. While, not as efficient in the transfer of KELEA as is paper, cotton may also serve as a general means of bringing KELEA into the body. Similarly, cotton bandages are widely used to dress wounds. Better results are now to be expected using paper bandages, especially if the paper bandages have been previously indirectly exposured to KELEA activated water.

Prior studies have shown ultraviolet fluorescence of the body's hairs in patients with a presumed insufficiency of cellular energy. It would not be surprising is keratin can also act in a manner similar to cellulose in plants as a natural receiver of KELEA. Melanin may also serve a similar function. As a constructed material, paper would seem to have significant advantages in attracting and transferring KELEA to life forms when compared with unprocessed natural materials. Indeed, the widespread availability of paper offers enormous benefits for its use as an additional means for increasing the amounts of KELEA entering into humans, animals, and plants. Paper apparently has a greater natural attraction for environmental KELEA than do humans or regular water. For reasons that are not presently understood, KELEA appears to more readily pass indirectly from paper to humans, than it passes indirectly from KELEA activated water to humans. Yet, KELEA activated water is an effective means of increasing the natural levels of KELEA in paper and, therefore, the paper's capacity to transmit KELEA to the body. Moreover, as noted above, paper with depleted KELEA can seemingly attract KELEA even from electromagnetic radiation. As a source of KELEA for the body, it is not necessary, therefore, that the KELEA levels in paper be heightened by the paper being first placed close to KELEA activated water. Fortunately, KELEA activated water can be easily prepared using a wide variety of methods.

There are now many clinical benefits of using KELEA activated water in human and animal health. For example, Enercel has been shown to add to the resistance to many infections, promote scar-free tissue regeneration, suppress tumor growth, and elevate the overall sense of wellness. Typically, Enercel is administered by intravenous injection, although it is also provided as an inhalant, sub-lingual dosage and skin spray. The Applicant has pioneered the use of Enercel as a wearable waterceutical, with promising results. The present indications, however, is that the use of paper with a heightened level of KELEA will likely perform as well as the direct use of Enercel in its many direct human and animal applications. Moreover, the use of paper with a heightened level of KELEA is far more economical since nothing is expended with its use. Thus, the bottles of activated water remain closed and the same sheets of paper are repeatedly used. Measuring the exact weight of the paper item can be used to monitor its KELEA level and a reduction in its weigh when applied to the body can serve as an indication of beneficial energy transfer being received by an individual.

The observations described in this Application will also assist in basic studies on KELEA and the ACE pathway. It is particularly noteworthy that electromagnetic radiation has now been shown to have potential additive as well as subtractive effects on the KELEA content of paper. The relationship of KELEA to cosmic rays and to gravity are of scientific interest and the present observations should assist in such studies.

The invention now being described, it will be apparent to one of skill in the art that the findings are reflective of an entirely new paradigm of almost unlimited possibilities. It will also spur new discoveries into the physics of KELEA and its role in Nature. The discovery will also help solidify the understanding of the ACE pathway.

1. A method of enhancing the alternative cellular energy (ACE) pathway in human and animal subjects, and thereby benefiting the subjects, comprising the placement onto the skin of the subject, a cellulose containing material in which part of the measured weight of the material is attributed to a heightened level of an attracted energy, which the Applicant refers to as KELEA, being an abbreviation for kinetic energy limiting electrostatic attraction; the placement of the material being for a sufficient time to allow for the transfer of KELEA to the subject, resulting in a lowering of the measured weight of the placed material and an improvement in the wellbeing of the subject.

2. A method of quantitatively assessing the amounts of KELEA transfer to a subject from a cellulose containing material that has been applied directly onto the skin of the subject, comprising the measuring of the decrease in the weight of the cellulose containing material as a result of its having been applied directly to the skin of the subject.

3. The method of increasing the KELEA transferring capacity of a cellulose containing material by prior locating the material in the vicinity of containers of KELEA activated water and/or other fluids and/or in the vicinity of KELEA attracting devices, wherein the amount of KELEA transferred to the cellulose containing material can be quantitatively assessed as an increase of up to approximately 1% of the base weight of the material.

4. The method of claim 1 in which the cellulose containing material comprises one or multiple sheets of paper in the form of regular writing paper, magazines, newspaper, or other forms of paper-based articles.

5. The method of claim 2 in which the cellulose containing material comprises one or multiple sheets of paper in the form of regular writing paper, magazines, newspaper, or other forms of paper-based articles.

6. The method of claim 3 in which the cellulose containing material comprises one or multiple sheets of paper in the form of regular writing paper, magazines, newspaper, or other forms of paper-based articles.

7. The method of claim 1 in which the cellulose containing material is placed by a human subject between the skin and the regular clothing worn during the day and/or during the night by the subject.

8. The method of claim 1 in which the cellulose containing material is used in place of a cotton bandage in the covering of a wound or other form of localized tissue injury.

9. The method of claim 3 in which the cellulose containing material is used in place of a cotton bandage in the covering of a wound or other form of localized tissue injury.

10. The method of claim 1 in which the cellulose containing material is used in the treatment of medical conditions attributed to an insufficiency of cellular energy.

11. The method of claim 3 in which the cellulose containing material is used in the treatment of medical conditions attributed to an insufficiency of cellular energy.

12. The method of claim 3 in which comparisons can be made as to the relative efficiencies of similar containers of different formulations of KELEA activated water and/or other fluids and of different KELEA attracting devices in their capacity to transfer KELEA to cellulose containing materials, as evidenced by the rate of the increase in weight and the upmost level of achieved weight increase by the cellulose containing material when placed in the vicinity of the different formulations of KELEA activated water and/or other fluids and of different KELEA attracting devices.
